(19) United States
(12) Patent Application Publication
(10) Pub. No.: US 2025/0191780 A1
(43) Pub. Date: Jun. 12, 2025

(54) ANOMALY DETECTION FOR IDENTIFYING EXPOSURE EVENTS FROM BASELINE MOLECULAR MEASUREMENTS IN HUMAN HEALTH

(71) Applicants: The Johns Hopkins University, Baltimore, MD (US); Central Intelligence Agency, Washington, DC (US)

(72) Inventors: Amanda W. Ernlund, New Market, MD (US); Daniel S. Berman, Silver Spring, MD (US); Kristopher D. Rawls, Silver Spring, MD (US); Luke C. Mullany, Catonsville, MD (US); Stanley Ta, Silver Spring, MD (US); Kenneth V. Bowden, Sparks, MD (US); Anissa N. Elayadi, Bethesda, MD (US); Sarah E. Herman, Elkridge, MD (US); Michael P. Gordon, Middletown, MD (US)

(73) Assignees: The Johns Hopkins University, Baltimore, MD (US); Central Intelligence Agency, Washington, DC (US)

(21) Appl. No.: 18/901,588

(22) Filed: Sep. 30, 2024

Related U.S. Application Data
(60) Provisional application No. 63/607,623, filed on Dec. 8, 2023.

(51) Int. Cl. G16H 50/50; G16H 50/70

(52) U.S. Cl. CPC G16H 50/50; G16H 50/70

(57) ABSTRACT

A computer implemented method, system, and non-transitory computer-readable device for implementing a generalized metadata generation system for omics data is provided. In some embodiments, a generalized reconstruction model may be trained to perform anomaly detection on an unlabeled omics feature vector. Various embodiments provide generalized anomaly detection that are agnostic to specific organs or exposure groups through aggregating and preprocessing omics data from disparate datasets. The generalized metadata generation system may then generate and assign an anomalous or non-anomalous label as metadata to improve computational interpretability of omics data.

### CROSS REFERENCE TO RELATED APPLICATIONS

This application claims benefit of U.S. Provisional Patent Application 63/607,623 filed on Dec. 8, 2023, which is incorporated by reference herein in its entirety.

### STATEMENT REGARDING FEDERALLY SPONSORED RESEARCH OR DEVELOPMENT

This invention was made with support from the United States Government, which has certain rights in the invention.

### BACKGROUND

As multi-omics technology evolves, large volumes of omics data have been collected and successfully applied in various contexts. For example, multi-omics data has assisted in identifying biomarkers for cancer detection in medical diagnostics fields. Multi-omics data has also been helpful in detecting the progression patterns of certain disorders such as Alzheimer's disease. Multi-omics has also seen success in various fields outside of medicine such as agriculture, where it has led to the development of resilient crop strains and the optimization of food production processes. Many public repositories have been established to address these diverse scenarios, including Gene Expression Omnibus (GEO) and The Cancer Genome Atlas (TCGA). However, these advancements introduce certain technological challenges in how data may be efficiently organized, indexed, and interpreted.

### BRIEF DESCRIPTION OF THE DRAWINGS

The accompanying drawings are incorporated herein and form a part of the specification.

FIG. 1 illustrates an example block diagram of a training pipeline for a generalized metadata generation system, according to some embodiments.

FIG. 2 illustrates an example metadata assignment to unlabeled omics feature vectors, according to some embodiments.

FIG. 3 illustrates an example principal component analysis (PCA) plot of omics data before applying a first preprocessing technique, according to some embodiments.

FIG. 4 illustrates an example PCA plot of omics data after applying a first preprocessing technique, according to some embodiments.

FIG. 5 illustrates example PCA plots of omics data before applying a second preprocessing technique, according to some embodiments.

FIG. 6 illustrates example PCA plots of omics data after applying a second preprocessing technique, according to some embodiments.

FIG. 7 illustrates an example label tabulation, according to some embodiments.

FIG. 8 illustrates an example flow diagram of a method, according to some embodiments.

FIG. 9 illustrates an example computer system useful for implementing various embodiments.

In the drawings, like reference numbers generally indicate identical or similar elements. Additionally, generally, the left-most digit(s) of a reference number identifies the drawing in which the reference number first appears.

### DETAILED DESCRIPTION

Disclosed herein are system, apparatus, device, method and/or computer program product embodiments, and/or combinations and sub-combinations thereof for processing an unlabeled omics feature vector, which may facilitate more efficient and informed computer systems.

As multi-omics technology evolves, the volume of omics data collected has grown exponentially. Many public repositories have been established to address diverse scenarios and applications. However, with the ever-growing complexity and scale of these datasets, multi-omics systems are facing certain technological challenges in how to efficiently process, organize, and interpret all the data. As used herein, “omics data” may refer to biological quantifications or characterizations of specific biomolecules or molecular processes within an organism. Omics data may include but is not limited to genomic data, proteomic data, transcriptomic data, metabolomics data, phenomic data, metabolomics data, and microbiomic data.

Generally, different omics repositories tend to tailor their data collection methods to specific research goals. For example, conventional pipelines may leverage datasets specific to a target diagnosis or diagnoses and develop diagnosis-specific models. These specialized models may then make determinations whether given data samples possess positive or negative diagnoses. While these models are useful for making determinations regarding the target diagnosis, they have low generalizability in making determinations regarding other diseases or simply whether the omics sample is healthy. In addition, these approaches require certain levels of a priori knowledge regarding the specific disease state and a corresponding healthy population to develop the model. As used herein, “a priori knowledge” may refer to knowledge gained previously, for example through previous clinical omics studies. Due to these requirements, entities undergo the inefficient processes of seeking out and enrolling patients with specific attributes (e.g. having/lacking the disease state) and manually labeling the data samples based on those specific attributes. Current systems are restricted by requiring a priori knowledge and are currently lacking in computational interpretability and translation. “Computational interpretability” may refer to how a computer or processing system understands, acknowledges, and/or organizes data.

Additionally, differences in experimental design and execution may also introduce technical noise or biases, resulting in data heterogeneity. These biases are problematic, as they obfuscate the true nature of the data samples, further contributing to the problems of computational interpretability and data infidelity. “Data fidelity” may refer to how accurately data reflects its source. “Data infidelity” may represent the problem where data does not accurately represent its source and may have been obfuscated due to experimental noise. Repository-specific noise and biases pose various technical challenges for endeavors aiming to integrate or compare datasets across disparate repositories. As such, these technical challenges hinder multi-omics systems from being able to fully exercise the potential of multi-omics data.

The technology described in the various embodiments herein implements a generalized metadata generation system for increasing the computational interpretability of an omics data sample. Improved computational interpretability may facilitate more informed and efficient computer processes. For example, improved computational interpretability may facilitate more robust computer indexing methods that may cut excess processing time and wasted resources. In another example, improved computational interpretability may enable context-aware data processing, such as metaheuristic or guided searching engines. In yet another example, improved computational interpretability may facilitate improved data compression systems (e.g. semantic data compression), allowing computer systems to perform the same functions by using less data, thereby improving computational efficiency and data storage implementations.

In some embodiments, the omics data sample may be an omics feature vector. In some embodiments, the generalized metadata generation system may first receive a plurality of datasets containing omics feature vectors. The generalized metadata generation system may then preprocess the plurality of datasets to produce a preprocessed dataset. In some embodiments, the generalized metadata generation system may label the omics feature vectors in each of the plurality of datasets as anomalous or non-anomalous. A non-anomalous (or non-exposed) label may denote that an omics feature vector is not associated with or has not been exposed to a disease, an illness, or adverse health symptoms. A non-anomalous label may also denote that an omics feature vector has not been exposed to some negative health event. In some embodiments, the generalized metadata generation system may remove technical noise across the plurality of datasets using one or more normalization techniques and aggregate the plurality of denoised datasets to create a generalized preprocessed dataset. The generalized metadata generation system may then separate the generalized preprocessed dataset into a subset of omics feature vectors labeled as non-anomalous and a subset of omics feature vectors labeled as anomalous. In some embodiments, the generalized metadata generation system may confirm that the technical noise across the plurality of datasets has been reduced using principal component analysis (PCA) or other dimensionality reduction techniques.

In some embodiments, the generalized metadata generation system may train a machine learning (ML) model using the subset of omics feature vectors labeled as non-anomalous to perform anomaly detection. The ML model may be an autoencoder or a convolutional neural network. The unlabeled omics feature vector may alternatively be a previously labeled feature vector whose label has been removed or dropped from consideration. The generalized metadata generation system may then provide an unlabeled omics feature vector to the trained ML model. The unlabeled omics feature vector may be obtained, for example, by processing data generated by analyzing a biological sample collected from an individual, such as a blood sample, non-invasive body fluid sample, or tissue sample. For example, physical RNA may be extracted from a biological sample through cell lysis and RNA precipitation. The RNA may then be converted to cDNA and measured for gene expression, e.g. through RNA sequencing or microarray analysis. A processing system may then align and quantify the reads or signal intensities of the cDNA with respect to target genes. An unlabeled omics feature vector may be produced as a result. Other techniques for generating an unlabeled omics feature vector from properties and/or biomarkers exhibited by a biological sample may additionally or alternatively be used.

The generalized metadata generation system may then generate a low-dimensional latent space of the unlabeled omics feature vector using the trained ML model. From this latent space representation, the generalized metadata generation system may reconstruct the omics feature vector to produce a reconstructed feature vector using the trained ML model. In some embodiments, generalized metadata generation system may evaluate a reconstruction error between the original unlabeled omics feature vector and the reconstructed feature vector against an anomaly threshold to obtain a reconstruction error evaluation. The generalized metadata generation system may then generate a feature label indicating whether the omics feature vector is anomalous or non-anomalous based on the reconstruction error evaluation. The generalized metadata generation system may then assign the generated feature label to the omics feature vector as metadata.

This generalized approach provides a direct improvement over previous systems, which required a priori knowledge. For example, previous systems were required to identify a target disease status to which they compare new, unknown data. However, when no target disease is identified or known, previous systems are unable to generalize and provide useful determinations on the unknown data point. By removing the a priori knowledge requirement of a pre-existing disease state and simply training using the control population data (e.g. healthy, non-exposed, non-anomalous, etc.), this approach has at least the dual effects of saving computational resources (e.g. training using less data) while still remaining generalizable to data it was not trained on. As such, the efficiency and computational interpretability of omics processing systems may be improved as compared to prior systems.

In some embodiments, the ML model(s) may be trained using supervised or semi-supervised learning, for example, by providing a collection of homogenously labeled omics feature vectors (e.g., non-anomalous) to an untrained or partially trained model to train the ML model. Upon being provided the omics feature vectors, the ML model(s) may be configured to generate low-dimensional latent space representations of the omics feature vectors and reconstruct the omics feature vectors from their corresponding low-dimensional latent space representations. The ML model(s) may then calculate a reconstruction loss using the reconstructed omics feature vectors and provide a determination or a likelihood that the omics feature vectors are anomalous or non-anomalous.

ML involves computers discovering how they can perform tasks without being explicitly programmed to do so. ML may include, but is not limited to, artificial intelligence, deep learning, fuzzy learning, supervised learning, unsupervised learning, etc. Machine learning algorithms may build a model based on sample data, known as “training data,” in order to make predictions or decisions without being explicitly programmed to do so. For supervised learning, the computer may be presented with example inputs and their desired outputs and the goal is to learn a general rule that maps inputs to outputs. In another example, for unsupervised learning, no labels may be given to the learning algorithm, leaving it on its own to find structure in its input. Unsupervised learning can be a goal in itself (discovering hidden patterns in data) or a means towards an end (feature learning).

In some embodiments, a ML model or ML engine may continuously change weighting of model inputs to increase accuracy of the ML model(s). For example, weighting of specific data fields or model layers may be continuously modified in the model to trend towards greater accuracy or lower reconstruction loss, where reconstruction loss is recognized by how accurately the ML model reconstructs an input non-anomalous omics feature vector and accuracy is recognized by correct determinations of whether an omics feature vector is anomalous or non-anomalous. Conversely, term weighting that lowers accuracy or increases reconstruction loss may be lowered or eliminated.

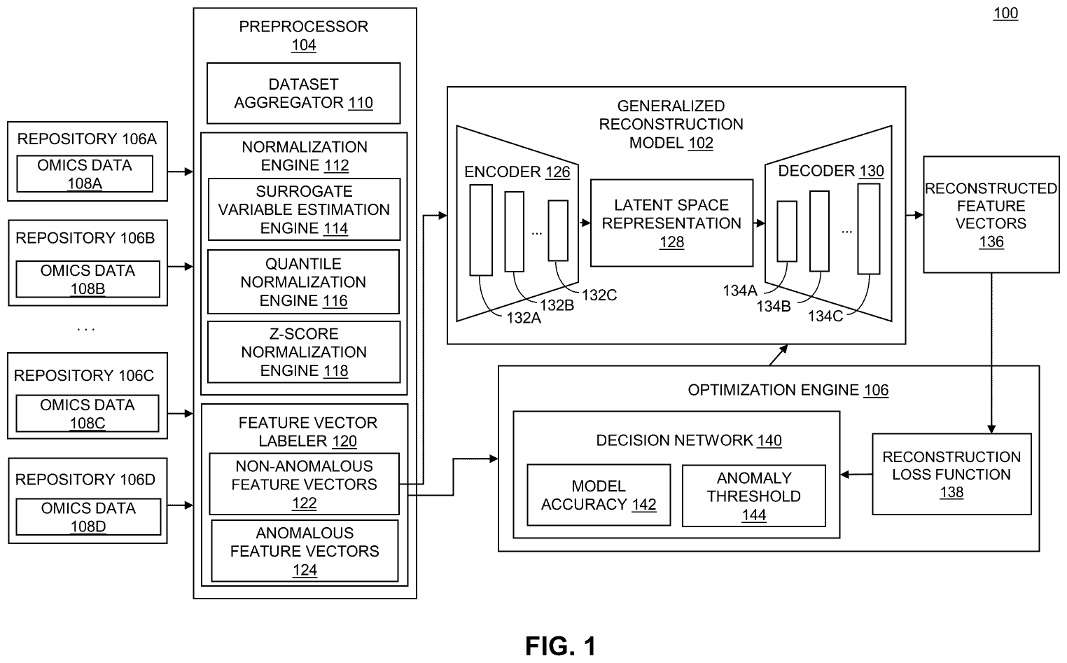

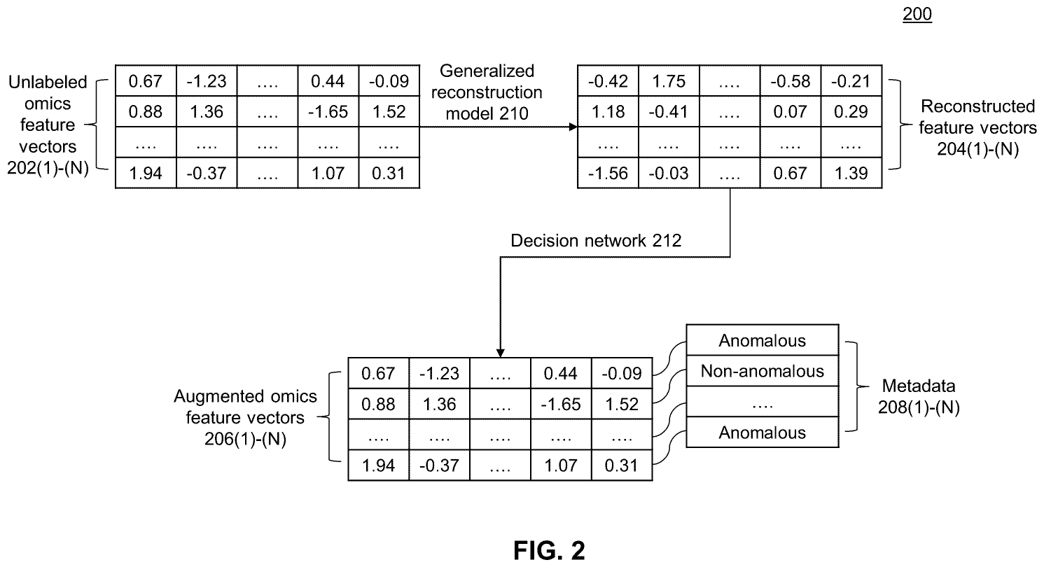

Various embodiments of this disclosure may be implemented using and/or may be part of a generalized metadata generation system shown in FIGS. 1-2. It is noted, however, that this environment is provided solely for illustrative purposes, and is not limiting. Embodiments of this disclosure may be implemented using and/or may be part of environments different from and/or in addition to the generalized metadata generation system, as will be appreciated by persons skilled in the relevant art(s) based on the teachings contained herein.

An example of the generalized metadata generation system shall now be described.

FIG. 1 illustrates an example generalized metadata generation training pipeline 100, according to some embodiments. Operations described may be implemented by processing logic that may comprise hardware (e.g., circuitry, dedicated logic, programmable logic, microcode, etc.), software (e.g., instructions executing on a processing device), or a combination thereof. It is to be appreciated that not all operations may be needed to perform the disclosure provided herein. Further, some of the operations may be performed simultaneously, or in a different order than described for FIG. 1, as will be understood by a person of ordinary skill in the art.

Generalized metadata generation training pipeline 100 illustrates the process of training generalized reconstruction model 102. In some embodiments, generalized metadata generation training pipeline 100 may operate partially or entirely within a generalized metadata generation system. Alternatively or additionally, in some embodiments, generalized metadata generation 100 may operate partially or entirely at third party servers or within the cloud.

As shown in FIG. 1, generalized metadata generation training pipeline 100 may include multiple repositories such as but not limited to repository 106A, repository 106B, repository 106C, and repository 106D. Repositories 106A-D may be public repositories, including but not limited to the Genotype Tissue Expression (GTEx) Project, The Cancer Genome Atlas (TCGA), the Encyclopedia of DNA Elements (ENCODE) data portal, Gene Expression Omnibus (GEO), Genomic Data Commons (GDC) data portal, and Omics Discovery Index (ODI). One or more of repositories 106A-D may also be datasets generated from data samples or clinical trials. In some embodiments, repositories 106A-D may include corresponding omics data 108A-D. Omics data 108A-D may include gene expression levels or methylation statuses of mRNA, miRNA, methylated DNA, or microbiomes. Alternatively or in addition, omics data 108A-D may include DNA sequences, single nucleotide polymorphisms (SNPs), single nucleotide variants (SNVs), insertion-deletions, chromatin accessibility (ATAC-seq data), and protein expression levels. In some embodiments, omics data 108A-D may come pre-labeled. For example, omics data 108A-D may include a label features indicating a tissue of origin (e.g. brain, lung, heart, etc.) and a disease or exposure status (e.g. cancer, asthma, COVID, smoking, healthy/control, etc.). In some embodiments, omics data may be stored in any known file format, for example, as a GCTX, TSV, FPKM/FPKM-UQ, SOFT, BAM/SAM, BED, GTF/GFF, FASTQ, VSF, or HTSqe-Counts file.

As shown in FIG. 1, generalized metadata generation training pipeline 100 may also include preprocessor 104. Preprocessor 104 may be used to preprocess omics data 108A-D before omics data 108A-D passes through generalized reconstruction model 102. In some embodiments, preprocessor 104 may operate using resources from a cloud or third party server. Preprocessor 104 may include dataset aggregator 110. In some embodiments, dataset aggregator 110 may aggregate omics data 108A-D from different repositories 106A-D to create an aggregated dataset. For example, preprocessor 104 may align mRNA gene expression level features across omics data 108A-D and append omics data 108A-D with each other.

In some embodiments, preprocessor may also include normalization engine 112. Normalization engine 112 may perform one or more normalization techniques that can be applied to omics data 108A-D or an aggregated dataset. In some embodiments, normalization engine 112 may include surrogate variable estimation (SVE) engine 114. SVE engine 114 may be employed by normalization engine 112 to remove a variety of noise, including batch effects and other unwanted variation that may result from high-throughput experiments. As used herein, “batch effects” may refer to groups of measurements with qualitatively different behaviors across different experimental design and execution. For example, batch effects may occur if one group of experiments was performed on a Wednesday and another group of experiments was performed on a Sunday. A batch effect may also occur if different sets of equipment were used across different experiments. As such, SVE engine 114 may remove certain batch effects across multiple repositories with different experimental design and execution, e.g. repositories 106A-D.

In some embodiments, SVE engine 114 may first fit a mathematical model based on a variable of interest (e.g. disease state or healthy/control). SVE engine 114 may then calculate the residuals of the model, which may represent differences between the true observed omics data values and the model prediction values. SVE engine 114 may then perform singular value decomposition (SVD) on the residuals matrix to find any patterns of variation. These patterns of variation may be quantified impacts of any sources of batch effects mentioned previously, such as different experiment dates, different technicians, different equipment used, or any other unknown factors that may cause variation across different repositories. SVE engine 114 may then identify and retain the variation patterns that are least associated with the variables of interest through a selection process, such as but not limited to F-testing. The least associated variation patterns may represent the potential sources of batch effects (as opposed to the variables that actually contribute to the variable of interest).

In some embodiments, for each of these retained variation patterns, SVE engine 114 may also identify any highly correlated genes and create a corresponding compressed dataset. SVE engine 114 may apply SVD once more to each compressed dataset to obtain the corresponding surrogate variable that captures the respective variation pattern. In some embodiments, SVE engine 114 may stop at this step and return the obtained surrogate variables.

In some embodiments, SVE engine 114 may perform further preprocessing to narrow down the meaningful surrogate variables and filter out surrogate variables that may just be random noise. To accomplish this, SVE engine 114 may create a null distribution of surrogate variables and only keep the surrogate variables whose eigenvalues exceed a certain threshold (e.g. 95^{th} percentile) of the null distribution. By doing this, SVE engine 114 may ensure that only the surrogate variables representing patterns stronger than expected randomness are kept (and thereby remove any surrogate variables that may have appeared “by chance” due to naturally occurring noise). To create this null distribution, SVE engine 114 may first randomly permute the rows of the original dataset and re-perform the surrogate variable estimation process outlined above on the permutated dataset, producing another set of surrogate variables and corresponding eigenvalues. SVE engine 114 may then repeat this process any number of times (e.g. 1000 times) to produce a null distribution of eigenvalues.

In some embodiments, after identifying the meaningful surrogate variables, SVE engine 114 may then normalize omics data 108A-D or the aggregated dataset by regressing out the effects of the identified surrogate variables. For example, SVE engine 114 may calculate adjusted gene expression values by subtracting the component of each expression value that is attributable to the surrogate variables. As a result, SVE engine 114 may then produce a dataset or datasets containing less batch effects. Alternatively or in addition, SVE engine 114 may apply additional methods to remove batch effects, including but not limited to ComBat and RUVSeq.

In some embodiments, preprocessor 104 may also or alternatively include quantile normalization engine 116. Quantile normalization engine 116 may further assist in removing technical noise from omics data 108A-D. In some embodiments, quantile normalization engine 116 may remove certain technical noise within a repository (e.g. omics data 108A of repository 106A), such as batch effects across tissue feature vectors (e.g. bladder, cervix, kidney, lung, etc.), by forcing the distribution of gene expression values to be the same across all feature vectors. Quantile normalization engine 116 may operate under the assumption that total gene expression values should be equal across all omics feature vectors (but individual genes may still be expressed differently across different samples).

In some embodiments, to force the same distribution, quantile normalization engine 116 may first rank or sort the gene expression values of each feature vector. Quantile normalization engine 116 may then calculate the mean for each rank across all the feature vectors. Finally, quantile normalization engine 116 may replace the original gene expression values in each sample with the calculated mean values. As a result, all the samples will have the same numerical set of gene expression values (and thus the same distribution), but the original gene expression rankings are still maintained, thereby removing inter-sample noise.

In some embodiments, preprocessor 104 may also include z-score normalization engine 118. Z-score normalization engine 118 may further assist in removing technical noise from omics data 108A-D. In some embodiments, z-score normalization engine 118 may remove any noise or biases across features (e.g. the expressed genes). Z-score normalization engine 118 may standardize the gene expression values across samples, for example, by setting the mean to 0 and the standard deviation to 1.

In some embodiments, preprocessor may include feature vector labeler 120. Feature vector labeler 120 may perform a feature labeling process for each feature vector in omics data 108A-D or an aggregated dataset by assigning a non-anomalous label or an anomalous label. In some embodiments, a non-anomalous label may indicate that an omics data sample belongs to a healthy or control population. A non-anomalous label may alternatively indicate that a feature vector represents a non-exposed data sample. For example, a non-exposed data sample may refer to a data sample that is not associated with or has not been exposed to a disease, illness, or adverse health symptom. Conversely, an anomalous label may indicate that a feature vector belongs to an unhealthy population. An anomalous label may alternatively or additionally indicate that a feature vector represents an exposed data sample. For example, an exposed data sample may refer to a data sample that is associated with or has been exposed to a disease, illness, or adverse health symptom.

In some embodiments, omics data 108A-D may come pre-labeled by repositories 106A-D. For example, a repository (e.g. repository 106A) may denote whether a feature vector corresponds to a control group or an exposed asthma group inside a feature or metadata. In this example, feature vector labeler 120 may assign the label of anomalous to any feature vectors corresponding to the asthma group and non-anomalous to any feature vectors corresponding to the control group. In another non-liming example, a repository (e.g. 106B) may not indicate that a feature vector belongs to any particular group. Instead, the repository itself may be only associated with one group of feature vectors (e.g. a cancer group). In this example, feature vector labeler 120 may then assign the label of anomalous to all of the feature vectors in this repository.

In some embodiments, feature vector labeler 120, after performing an initial feature labeling process, may separate a labeled aggregated dataset into non-anomalous feature vectors 122 and anomalous feature vectors 124. For example, non-anomalous feature vectors 122 may include any combination or subcombination of the feature vectors across omics data 108A-D labeled as non-anomalous. Similarly, anomalous feature vectors 124 may include any combination or subcombination of the feature vectors across omics data 108A-D labeled as anomalous. Non-anomalous feature vectors 122 may represent a generalized healthy population across different repositories and different tissue sample groups (e.g. repositories 106A-D). Alternatively, non-anomalous feature vectors 122 may represent a generalized healthy population across different repositories but for one specific tissue sample group.

In some embodiments, preprocessor 104 may employ any combination or subcombination of the aforementioned preprocesing techniques. Alternatively or in addition, this disclosure contemplates using other types of normalization or preprocessing techniques to compensate for any batch effects and/or further assist in providing increased computational interpretability to omics data 108A-D. For example, preprocessor 104 may include a noisifying module (not shown) that may artificially add noise (e.g. Gaussian noise) to non-anomalous feature vectors 122 and/or anomalous feature vectors 124. Artificially adding noise to the feature vectors may allow the feature vectors to better reflect the random noise that exists in real world data, thereby increasing the robustness of any model that is trained using the noisified feature vectors. In some embodiments, the noisifying module may select a uniform noise intensity value to apply to all feature vectors. In some embodiments, noisifying module may select noise intensity values to apply to feature vectors at random. In some embodiments, the noisifying module may only select a subset of feature vectors to add noise to. In doing so, the noisifying module may generate diverse datasets that can properly reflect the diversity and nuance of real world data.

In some embodiments, generalized metadata generation training pipeline 100 may include generalized reconstruction model 102. Generalized reconstruction model 102 may be an autoencoder ML model that includes encoder 126 and decoder 130. Encoder 126 may include a neural network that transforms an input (e.g. non-anomalous feature vectors 122) to a corresponding latent space representation 128 through an encoding process. Decoder 130 may include a neural network that transforms a latent space representation 128 to a corresponding reconstructed output (e.g. reconstructed feature vectors 136) through a decoding process.

Encoder 126 may include one or more layers 132A-C, such as an input layer and one or more linear layers that are configured to extract core features from an input and transform higher dimensional inputs to lower dimensional latent space representations, with the final linear layer producing latent space representation 128. Each linear layer may also include a non-linear activation function, including but not limited to a softmax, softsign, softplus, swish, sigmoid, hyperbolic tangent, rectified linear unit (ReLU), leaky ReLU, parametric ReLU, and exponential linear unit (ELU). In a non-limiting example, encoder 126 may include an input layer with 16,432 nodes, where each node in the input layer may map to a gene expression level feature. In this non-limiting example, encoder 126 may also include four linear layers consisting of 1024 nodes, 512 nodes, 128 nodes, and 64 nodes respectively, each layer being followed by a ReLU activation function.

Decoder 130 may include one or more layers 134A-C, such as one or more intermediate linear layers that are configured to reconstruct higher dimensional estimations from a low-dimensional latent space representation (e.g. latent space representation 128) and are followed by non-activation functions. Layers 134A-C may also include a final decoder layer that produces the final high-dimensional estimation (e.g. reconstructed feature vectors 136). Continuing the previous non-limiting example, decoder 130 may include three linear layers that mirror encoder 126 and consist of 128 nodes, 512 nodes, and 1024 nodes respectively, each layer also being followed by a ReLU activation function. Decoder 130 may also include a final decoder linear layer with 16,432 nodes.

Generalized reconstruction model 102 may be trained, for example, on measurements as discrete molecule classes and/or as integrated measurements. In some embodiments, non-anomalous feature vectors 122 may be provided to an untrained or partially trained generalized reconstruction model 102 to further train generalized reconstruction model 102. By training using aggregated non-anomalous feature vectors 122, generalized reconstruction model 102 may provide generalized determinations that are agnostic to any particular tissue group(s) or exposure group(s). Doing so provides a technical improvement over prior prediction systems, which have primarily been developed using specific tissue group(s) and/or exposure group(s) and are thus not incapable of making agnostic determinations.

An “untrained or partially trained” model may refer to a completely untrained model (i.e. a model with default weight values) or a model that has received some training (i.e. a model with partially updated weight values). Any level of training may be considered “partially trained” if a model can be further trained to fine-tune the model, for example, to better perform a specific task. An example of a partially trained ML model may be a pre-trained ML model. In some embodiments, a pre-trained generalized reconstruction model generated by a third party may be obtained and trained using transfer learning to repurpose the pre-trained generalized reconstruction model to the generalized metadata generation process described herein. The transfer learning may be conducted on an ML platform and may involve training as disclosed herein.

“Further train,” “further trained,” or “further training” should not be interpreted to mean that a model has already been at least partially trained before being “further trained.” Rather, “further train,” “further trained,” or “further training” may refer to an ML model that was partially trained and has now undergone additional training, or may refer to an ML model that was entirely untrained and has undergone some training. Accordingly, “further trained” indicates that a model receives additional training, refinement, updating, etc., than it previously had. Likewise, “train,” “training,” or “trained” should not be interpreted to mean in all cases that a model is fully trained (e.g., using a particular method) and cannot be refined or updated further, but only that some amount of training is being or has been performed (e.g., using a particular method).

In some embodiments, generalized metadata generation training pipeline 100 may include optimization engine 106. Optimization engine 106 may train or further train generalized reconstruction model 102 by optimizing the internal weights of generalized reconstruction model 102 against one or more loss functions. In some embodiments, the loss function may include reconstruction loss (e.g. reconstruction loss function 138). Reconstruction loss may refer to the difference between the final reconstructed feature vectors 136 and the corresponding non-anomalous feature vectors 122. In some embodiments, reconstruction loss may be calculated by using mean-squared error. In some embodiments, the mean-squared reconstruction error may be transformed, for example, using a log base 10 transformation. In some embodiments, reconstruction loss may be calculated in other ways, such as but not limited to mean absolute error, Huber loss, log-cosh loss, and Poisson loss.

While reconstruction loss functions are discussed herein, other types of loss functions are contemplated. For example, the one or more loss functions may include perceptual loss and/or adversarial loss. Perceptual loss may measure the difference between high-level (e.g. inter-gene) biological features of non-anomalous feature vectors 122, rather than individual gene-level differences as in the case with reconstruction loss. In some embodiments, perceptual loss may be calculated using a pre-trained neural network. Including perceptual loss during training may be beneficial, as perceptual loss aims to capture and maintain perceptual and semantic details from a low-dimensional latent space representation.

Alternatively or in addition, the one or more loss functions may include adversarial loss. For example, a discriminating module may be a separate classifier model trained to distinguish between the reconstructed feature vectors 136 and the original non-anomalous feature vectors 122. The discriminating module may be trained alongside generalized reconstruction model 102 and may provide feedback to generalized reconstruction model 102 on how realistic the reconstructed feature vectors 136 are. The more realistic the reconstructed feature vectors 136 produced by generalized reconstruction model 102, the harder it will be for the discriminating module to distinguish reconstructed feature vectors 136 from original non-anomalous feature vectors 122, and thus adversarial loss may be minimized. By minimizing adversarial loss, generalized reconstruction model 102 may produce better quality reconstructed feature vectors 136 that more closely model the training distribution.

After selecting which one or more loss functions to use, optimization engine 106 may update the internal weights of one or more layers 132A-C and 134A-C of encoder 126 and decoder 130 using backpropagation and optimization algorithms such as, but not limited to, stochastic gradient descent, Adam, Nesterov Accelerated Gradient, root-mean square propagation, Adadelta, and Adamax. As a non-limiting example, optimization engine 106 may perform an iterative training process over 20 epochs using the Adam optimizer with a batch size and learning rate of 0.001. As used herein, an “epoch” may refer to a complete pass of a training dataset through a machine learning algorithm. “Batch size” may refer to the number of samples used during each iteration of a model's training process. “Learning rate” may determine how much internal weights are adjusted at each iteration. After the training process, the final learned weights may capture the meaningful patterns and structures in non-anomalous feature vectors 122 and help reconstruct accurate reconstructed feature vectors 136.

In some embodiments, optimization engine 106 may also include decision network 140. Decision network 140 may include an anomaly threshold 144. In some embodiments, decision network 140 may take trained generalized reconstruction model 102 and calculate a reconstruction error for each feature vector of non-anomalous feature vectors 122. Using these reconstruction errors, decision network 140 may construct a probability distribution (e.g. a normal distribution) and calculate an anomaly threshold 144. Anomaly threshold 144 may denote a reconstruction error cutoff value that decision network 140 may employ to determine whether a reconstruction error corresponds to an anomalous feature vector or a non-anomalous feature vector. For example, anomaly threshold 144 may have an a value of 0.0025, which signifies that any reconstruction error exceeding a 99.5% confidence interval may be labeled as anomalous by decision network 140. Conversely, any reconstruction error within the 99.5% confidence interval may be labeled as non-anomalous by decision network 140.

In some embodiments, optimization engine 106 may use anomaly threshold 144 to calculate model accuracy 142. For example, optimization engine 106 may employ generalized reconstruction model 102 to determine reconstruction errors of feature vectors within a testing dataset (e.g. a combination or subcombination of non-anomalous feature vectors 122 and anomalous feature vectors 124). Optimization engine 106 may then use anomaly threshold 144 to determine corresponding non-anomalous or anomalous feature labels for each reconstruction error. Optimization engine 106 may then tabulate and compare the determined and original labels to calculate model accuracy 142. In some embodiments, anomaly threshold 144 may be empirically determined. Alternatively, anomaly threshold 144 may be determined through an optimization process that maximizes model accuracy 142. For example, model accuracy 142 may be used in a multi-objective optimization function that attempts to maximize model accuracy 142 and minimize one or more loss functions (e.g. reconstruction loss function 138). In some embodiments, the resulting multi-objective optimization function may be solved using scalarization methods, evolutionary algorithms, decomposition methods, and the like.

FIG. 2 illustrates an example metadata assignment 200, according to some embodiments. Operations described may be implemented by processing logic that may include hardware (e.g., circuitry, dedicated logic, programmable logic, microcode, etc.), software (e.g., instructions executing on a processing device), or a combination thereof. It is to be appreciated that not all operations may be needed to perform the disclosure provided herein. Further, some of the operations may be performed simultaneously, or in a different order than described for FIG. 2, as will be understood by a person of ordinary skill in the art.

Example metadata assignment 200 may include unlabeled omics feature vectors 202(1)-(N), reconstructed feature vectors 204(1)-(N), augmented omics feature vectors 206(1)-(N), metadata 208(1)-(N), generalized reconstruction model 210, and decision network 212. In some embodiments, example metadata assignment 200 may provide unlabeled omics feature vectors 202(1)-(N) to a generalized reconstruction model 210 to generate one or more inferences of reconstructed feature vectors 204(1)-(N). Generalized reconstruction model 210 may be an example of generalized reconstruction model 102 (of FIG. 1) that has been trained. An “inference” may refer to the process of running new data points through a trained machine learning model to calculate outputs and/or predictions. For example, unlabeled omics feature 202(1) with normalized gene expression values [0.67, −1.23, . . . , 0.44, −0.09] may be reconstructed as reconstructed feature vector 204(1) with reconstructed gene expression values [−0.42, 1.75, . . . , −0.58, −0.21] using generalized reconstruction model 210.

Example metadata assignment 200 may then provide reconstructed feature vectors 204(1)-(N) to decision network 212 to generate augmented omics feature vectors 206(1)-(N). Decision network 212 may be an example of decision network 140 (of FIG. 1). In some embodiments, decision network 212 may first calculate a reconstruction error for each of reconstructed feature vectors 204(1)-(N). Decision network 212 may then generate feature labels indicating whether each of reconstructed feature vectors 204(1)-(N) are anomalous or non-anomalous by evaluating the corresponding reconstruction error values against an anomaly threshold. For example, decision network 212 may determine that the reconstruction error corresponding to reconstructed feature vector 204(1) with reconstructed gene expression values [−0.42, 1.75, . . . , −0.58, −0.21] lies outside the determined 99.5% confidence interval. As such, decision network 212 may generate an anomalous label for reconstructed feature vector 204(1). After generating anomalous or non-anomalous labels for reconstructed feature vectors 204(1)-(N), decision network 212 may assign the corresponding labels as metadata 208(1)-(N) to unlabeled omics features vectors 202(1)-(N) and generate augmented omics feature vectors 206(1)-(N).

The technical solution disclosed above allows for a generalized metadata generation for omics feature vectors to improve computational interpretability. This solution streamlines and enhances omics data organization and interpretability processes.

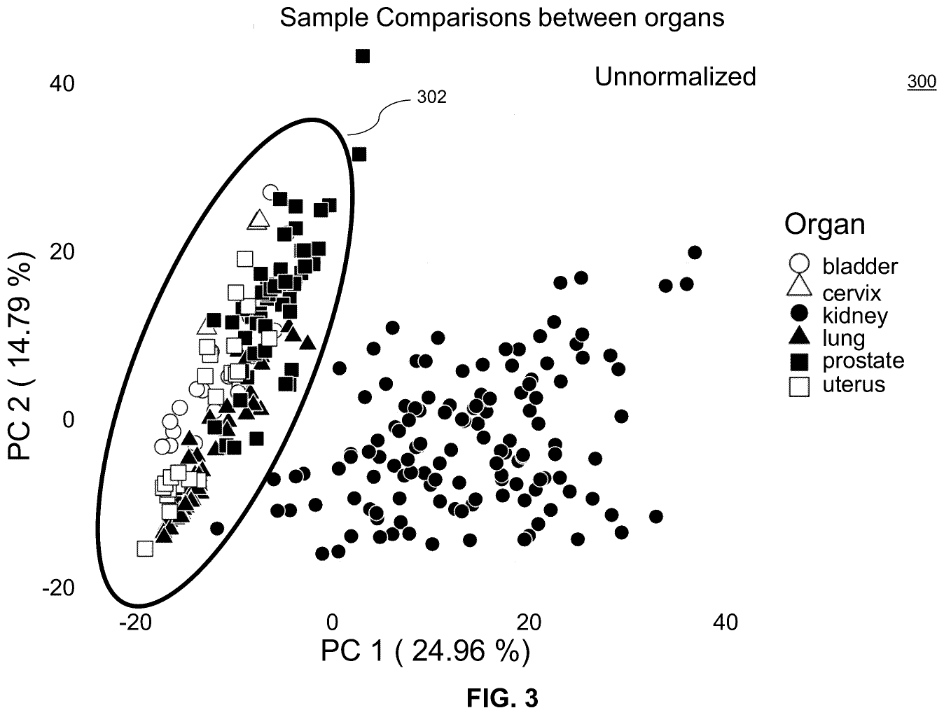

FIG. 3 illustrates an example principal component analysis (PCA) plot 300 of omics data before applying a first preprocessing technique, according to some embodiments. “PCA” may involve simplifying high-dimensionality data (e.g. gene expression omics data) into lower dimensional representations while preserving most of the data's structure. PCA may assist in visualizing labeled high dimensional data. For example, example PCA plot 300 may plot a set of omics feature vectors across multiple organ groups, such as the bladder, cervix, kidney, lung, prostate, and uterus within the same repository prior to normalization. Different organ groups may typically share similar gene expression levels within their respective class. This may typically also be reflected in a PCA plot, where similar classes tend to form clusters. However, technical noise may interfere with this natural clustering, such as in example PCA plot 300. In 302, there may not be any clear clusters for any specific organ group. For example, due to the technical noise, the current gene expression levels of feature vectors from the bladder, cervix, lung, prostate, and uterus organ groups may not be distinguishing enough to be reflected in example PCA plot 300. Only the kidney organ group may seem distinguishable from the other organ groups prior to any normalization methods.

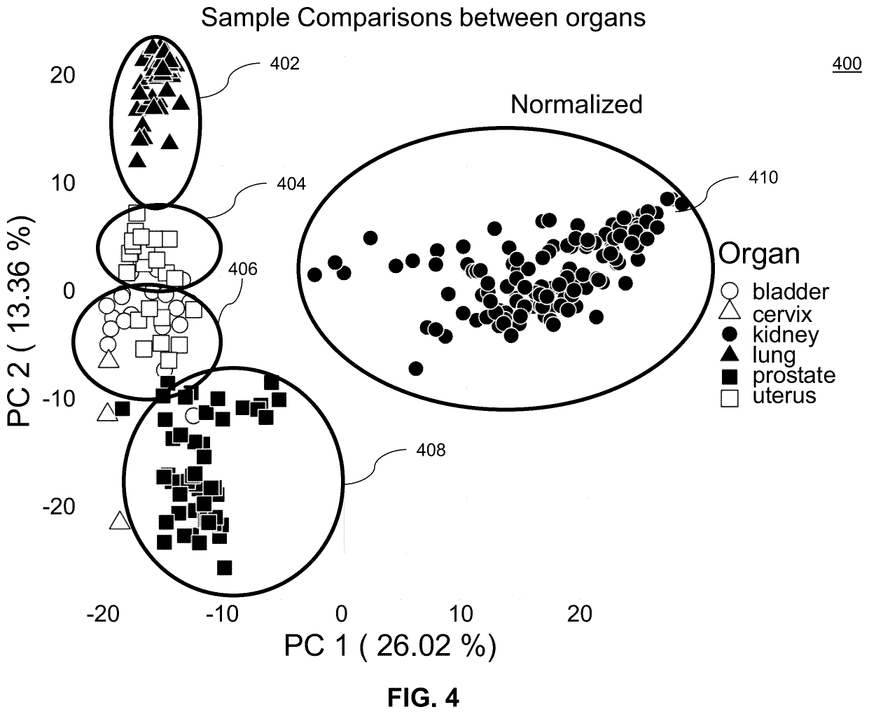

FIG. 4 illustrates an example PCA plot 400 of omics data after applying a first preprocessing technique, according to some embodiments. Example PCA plot 400 may plot a set of omics feature vectors across the same organ groups as the example PCA plot 300 of FIG. 3 and may represent a PCA plot after performing a first preprocessing technique. In some embodiments, the first preprocessing technique may be quantile normalization 116. As discussed in FIG. 1, quantile normalization 116 may assist in removing batch effects across tissue groups and/or different exposure groups within a repository. In 402, omics feature vectors corresponding to the lung organ group may form a cluster. In 404, omics feature vectors corresponding to the uterus organ group may form another cluster. In 406, omics feature vectors corresponding to the bladder organ group may form a third cluster. In 408, omics feature vectors corresponding to the prostate organ group may form a fourth cluster. In 410, omics feature vectors corresponding to the kidney organ group may remain in a cluster.

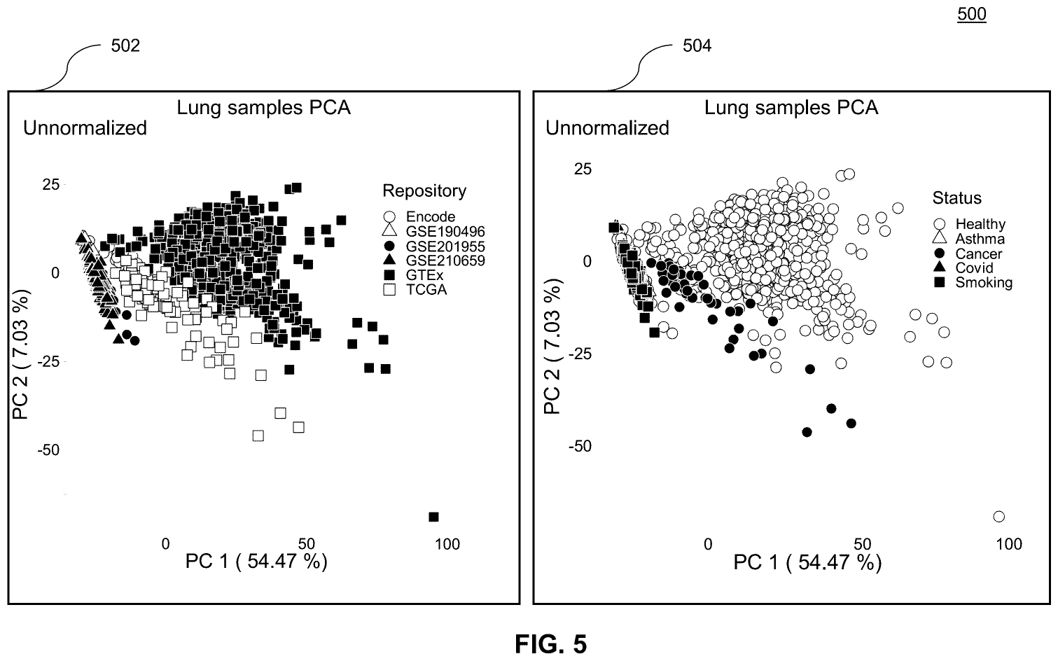

FIG. 5 illustrates example PCA plots 500 of omics data before applying a second preprocessing technique, according to some embodiments. In 502, a PCA plot for a set of omics feature vectors corresponding to the lung organ across multiple repositories (e.g. Encode, GSE190496, GSE201955, GSE210659, GTEx, and TCGA) may be depicted. In contrast to organ groups, different repositories may typically not be expected to form clusters. The gene expression levels should mostly depend on the biological markers of the organ group rather than the repository that conducted the data collection. As such, the clustering depicted in 502 may be due to the presence of batch effects that may hurt overall computational interpretability and data fidelity. In 502 and 504, quantile normalization engine may have already applied one or more quantile normalization techniques. As such, the clustering of different exposure groups (e.g. healthy/control, asthma, cancer, COVID, and smoking) depicted in 504 may be a positive indication that potential batch effects across exposure groups have been lessened.

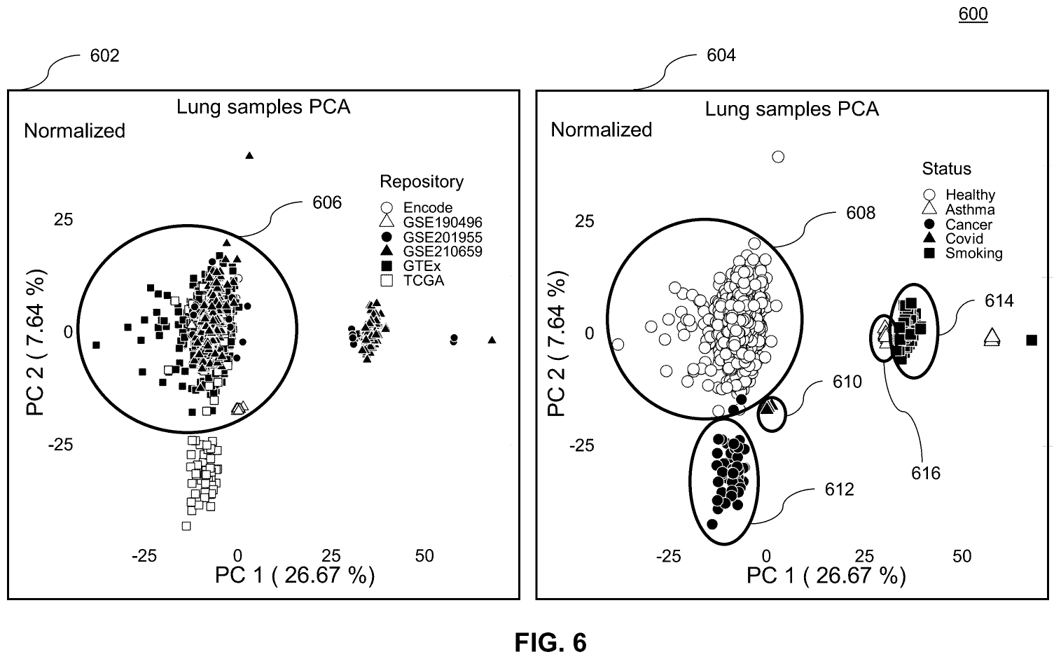

FIG. 6 illustrates example PCA plots 600 of omics data after applying a second preprocessing technique, according to some embodiments. In some embodiments, the second preprocessing technique may be SVE. For example, as discussed in FIG. 1, SVE engine 114 may assist in removing batch effects across different repositories through SVE techniques. In 602, a PCA plot for a set of omics feature vectors corresponding to the lung organ group across the same repositories as 502 of FIG. 5 may be depicted. In 606, there may not be any clear clusters for any repositories. This may suggest that the gene expression levels of the normalized omics feature vectors no longer contain batch effects that may interfere with computational interpretability and data fidelity. In 604, feature vectors within the same exposure group may still form clusters. For example, in 608, a cluster of healthy or control feature vectors may be depicted. In 610, a cluster of COVID feature vectors may be depicted. In 612, a cluster of cancer feature vectors may be depicted. In 614, a cluster of smoking feature vectors may be depicted. In 616, a cluster of asthma feature vectors may be depicted.

While specific PCA plots, organ groups, and exposure groups have been described herein, these examples are not meant to represent an exhaustive list of possible implementations. Rather, the examples depicted aim to illustrate the effectiveness of one or more preprocessing techniques described herein for improving computational interpretability and data fidelity. Therefore, the scope of the technology disclosed herein is not limited to only these examples.

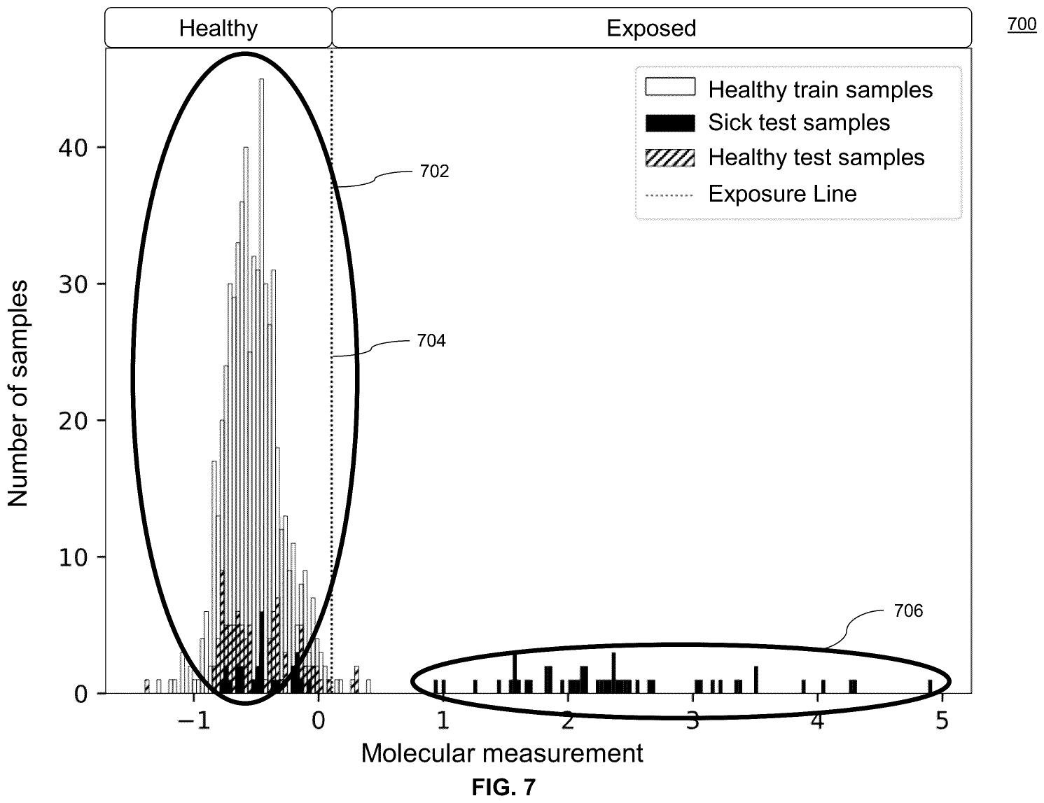

FIG. 7 illustrates an example label tabulation 700, according to some embodiments. For example, example label tabulation 700 may depict a tabulated set of labels generated by decision network 140 (of FIG. 1) using a trained generalized reconstruction model. Example label tabulation 700 may also include exposure line 704, which may reflect anomaly threshold 144. Exposure line 704 may represent the cutoff value for determining whether any given omics feature vector is assigned an anomalous or non-anomalous label. For example, in 702, the tabulated set of healthy labels may be depicted. In 706, the tabulated set of sick test samples may be depicted.

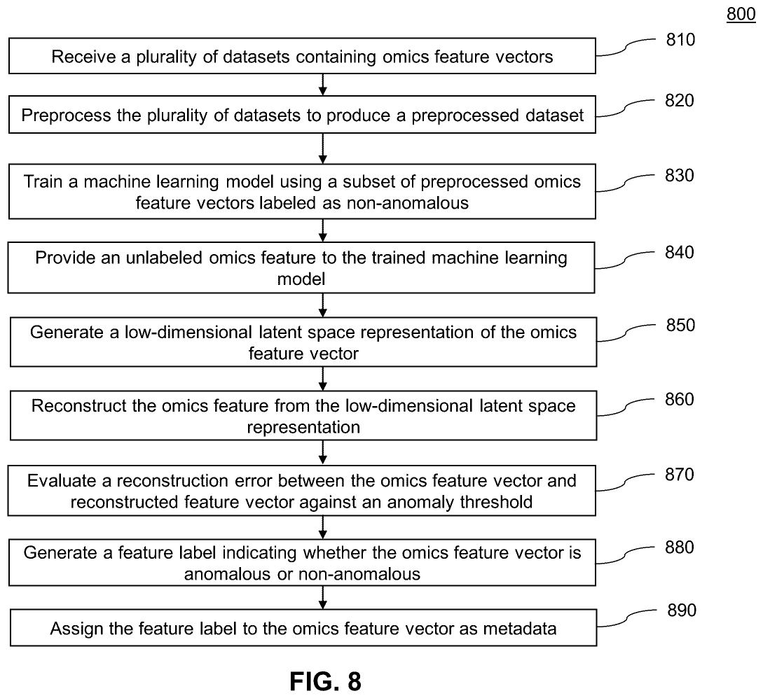

FIG. 8 is a flow chart depicting a method 800 that can be carried out in line with the discussion above. Method 800 shall be described with reference to FIGS. 1-2. However, method 800 is not limited to those example embodiments. One or more of the operations in the method depicted by FIG. 8 may be carried out by one or more entities, including, without limitation, an edge server or cloud-based server processing systems and/or one or more entities operating on behalf of or in cooperation with these or other entities. Any such entity may embody a computing system, such as a programmed processing unit or the like, configured to carry out one or more of the method operations. Further, a non-transitory data storage (e.g., disc storage, flash storage, or other computer readable medium) may have stored thereon instructions executable by a processing unit to carry out the various depicted operations. In some embodiments, the systems described train and implement a generalized metadata generation system for improving computational interpretability, organization, and efficiency of multi-omics systems.

Unless stated otherwise, the steps of method 800 need not be performed in the order set forth herein. Additionally, unless specified otherwise, the steps of method 800 need not be performed sequentially. The steps may be performed in a different order or simultaneously. Further, method 800 may not include all the steps illustrated. For example, in some embodiments, method 800 may not include steps 810-830. In some embodiments, method 800 may not include steps 810-830, for example, if generalized metadata generation system 100 has been previously trained or is obtained from an external source.

Step 810 may include receiving a plurality of datasets containing omics feature vectors. For example, generalized metadata generation system 100 may receive omics data 108A-D from one or more repositories (e.g. repositories 106A-D). Omics data 108A-D may come from public repository datasets or generated datasets and may include, but are not limited to, feature vectors of gene expression levels or methylation statuses of mRNA, miRNA, methylated DNA, and microbiomes.

Step 820 may include preprocessing the plurality of datasets to produce a preprocessed dataset. For example, generalized metadata generation system 100 may employ one or more preprocessing and normalization techniques to remove various technical noise and biases from the omics feature vectors. In some embodiments, preprocessor 104 may apply quantile normalization engine 116 to omics data 108A-D. Preprocessor 104 may then aggregate the preprocessed omics data 108A-D to produce an aggregated dataset. In some embodiments, preprocessor may then leverage SVE engine 114 and z-score normalization engine 118 to further preprocess the aggregated dataset. In some embodiments, preprocessor 104 may then employ feature vector labeler 120 to generate and assign anomalous or non-anomalous labels for each feature vector in the aggregated dataset. Preprocessor 104 may then partition the labeled aggregated dataset into non-anomalous feature vectors 122 and anomalous feature vectors 124. In some embodiments, the generalized metadata generation system may confirm that the technical noise across the plurality of datasets has been removed using PCA.

Step 830 may include training a machine learning model using a subset of preprocessed omics feature vectors labeled as non-anomalous to perform anomaly detection. For example, generalized metadata generation system 100 may provide non-anomalous feature vectors 122 to generalized reconstruction model 102. In some embodiments, generalized reconstruction model 102 may be an autoencoder or neural network. Optimization engine 106 may train or further train generalized reconstruction model 102 by optimizing the internal weights of generalized reconstruction model 102 against one or more loss functions. In some embodiments, the loss function may include reconstruction loss function 138. During the training process, optimization engine 106 may update the internal weights of one or more layers 132A-C and 134A-C of encoder 126 and decoder 130 using backpropagation and optimization algorithms. In some embodiments, decision network 140 may also determine an anomaly threshold 144 to use for anomaly detection.

Step 840 may include providing an unlabeled omics feature vector to the trained machine learning model. For example, example metadata assignment 200 may provide unlabeled omics feature vector 202(1) to generalized reconstruction model 210. Unlabeled omics feature vector 202(1) may be obtained, for example, by processing data generated by analyzing a biological sample collected from an individual, such as a non-invasive tissue sample.

Step 850 may include generating, using the trained machine learning model, a low-dimensional latent space representation of the omics feature vector. For example, generalized reconstruction model 210 may generate a low-dimensional latent space representation of unlabeled omics feature vector 202(1) using an encoder. Generalized reconstruction model 210 may apply one or more transformations to unlabeled omics feature vector 202(1) to reduce dimensionality while retaining high-level features.

Step 860 may include reconstructing, using the trained machine learning model, the omics feature vector from the low-dimensional latent space representation to produce a reconstructed feature vector. For example, generalized reconstruction model 210 may reconstruct the low-dimensional latent space presentation of unlabeled omics feature vector 202(1) into reconstructed feature vector 204(1) using a decoder. Generalized reconstruction model 210 may apply one or more transformations to unlabeled omics feature vector 202(1) to increase dimensionality and reconstruct omics feature vector 202(1) according to learned internal weights.

Step 870 may include evaluating a reconstruction error between the omics feature and the reconstructed feature vector against an anomaly threshold to obtain a reconstruction error evaluation. For example, example metadata assignment 200 may provide reconstructed feature vector 204(1) to decision network 212. In some embodiments, decision network 212 may employ a reconstruction loss function to calculate a corresponding reconstruction loss between unlabeled omics feature vector 202(1) and reconstructed feature vector 204(1). Decision network 212 may then evaluate the calculated reconstruction loss against an anomaly threshold to obtain a reconstruction error evaluation.

Step 880 may include generating a feature label indicating whether the omics feature vector is anomalous or non-anomalous based on the reconstruction error evaluation. For example, if the reconstruction error evaluation signifies that the reconstruction loss calculated by decision network 212 lies outside a confidence interval defined by the anomaly threshold, decision network 212 may generate an anomalous label for unlabeled omics feature vector 202(1). If the reconstruction error evaluation signifies that the reconstruction loss lies within a confidence interval defined by the anomaly threshold, decision network 212 may generate a non-anomalous label for unlabeled omics feature vector 202(1).

Step 890 may include assigning the feature label to the omics feature vector as metadata. For example, decision network 212 may augment unlabeled omics feature vector 202(1) with metadata 208(1) associated with an anomalous label to generate augmented omics feature vector 206(1).

The solutions described above provide technical solutions to shortcomings of current omics data processing systems. The various embodiments solve at least the technical problems associated with computational interpretability, organization, and generalizability of current omics data processing systems through generalized metadata generation, resulting in a more efficient organization and processing of omics systems.

The various embodiments encompassed by the technology disclosed herein are able to generate and assign accurate and generalizable metadata that effectively capture biological patterns across disparate repositories and datasets. This generalized approach provides a direct improvement over previous systems, which required a priori knowledge. For example, previous systems were required to identify a target disease status to which they compare new, unknown data. However, when no target disease is identified or known, previous systems are unable to generalize and provide useful determinations on the unknown data point. By removing the a priori knowledge requirement of a pre-existing disease state and simply training using the control population data (e.g. healthy, non-exposed, non-anomalous, etc.), this approach has at least the dual effects of saving computational resources (e.g. training using less data) while still remaining generalizable to data it was not trained on. As such, the efficiency and computational interpretability of omics processing systems may be improved as compared to prior systems.

### Example Experimental Use Case—mRNA to Distinguish Exposure from Normal Tissue

### Preprocessing

Accurate detection of an exposure or disease event is currently a challenge in the field of diagnostics. Biomolecular biomarkers may be used to distinguish different disease states from healthy and can be used to define disease states. Biomarkers include the suite of proteins, small molecules, RNA, and epigenetic markers on DNA that change across healthy and disease. To identify biomarkers using current tools, there must be a priori knowledge of the specific disease state and a defined healthy population. For every particular disease, biomarkers must be discovered through clinical studies of people with the disease. These studies can suffer from a lack of generalizability of biomarkers to a new cohort of individuals and can be underpowered due to cost and difficulty in recruiting patient populations.

To address these challenges, and in accordance with a specific experimental use case incorporating aspects of the above disclosure, the inventors developed an analytics pipeline to detect healthy from exposed individuals using molecular biomarkers. This pipeline allows 1) early detection of exposure events in non-invasive samples without training the model on disease-specific data, 2) flexibility in the type of biomolecule utilized for detection, and 3) identification of exposure-affected tissue from biomolecular data. In this experimental use case, the model was trained using mRNA, miRNA, or methylation data curated from the public domain. While not part of the experiment conducted, a skilled artisan would recognize that small molecule metabolite data and protein data may also be used to train the model.

Biomarkers including mRNA, miRNA, methylation, and proteins are both tissue-specific and informative of disease states and exposure. Omics measurements, or genome-wide measurement of biomarker classes, captures expression of all molecules of that class in humans. Organs and organs systems will express particular genes consistently that function to maintain critical systems that allow for particular organ function. Due to tissue specificity of biomarker expression, several classes of biomarkers have been utilized for forensic purposes to identify body parts found as part of crime scenes. Further, biomarkers associated with particular organs and cancers have been identified from blood samples and may be a potential early diagnostic for disease. mRNA, miRNA, protein, and methylation are informative biomarkers that have the potential to be collected from non-invasive samples.

Tight regulation of cellular process including gene expression and epigenetic regulation cause biomarker levels to change in response to the environment. When people are exposed to disease, environmental stress, or lifestyle changes, gene expression allows for the body to produce a specific suite of protein effectors that modulate immune response, metabolism, cellular growth, and other physiological processes. Genome-wide omics studies of individuals with various health conditions have been performed over the last twenty years and studies have published data and analysis to public repositories for future study. Studies tend to focus on one disease/exposure and one tissue type.

For the purposes of utilizing omics to identify biomarkers for health anomaly detection, public data was aggregated across studies for identification of biomarkers of particular exposures and for potential to identify biomarkers common to exposures. In particular, to utilize different molecule classes including mRNA, miRNA, methylation, small molecules and proteins to detect exposure in a population and to identify organs affected by exposure, the inventors curated data from public repositories with a focus on identification of measurements from healthy individuals. Further, studies were collected for various exposures. Current data was curated from organs/tissues, blood, and other non-invasive samples types. To allow for comparison across studies, several methods for data normalization and batch removal were evaluated. An initial algorithm framework for anomaly detection was trained on healthy mRNA expression, miRNA expression or methylation levels. A threshold for anomalous biomarker signatures was determined for each class of molecule. The algorithm showed initial success in segmenting healthy individuals from exposed individuals. To identify biomolecules specific to tissue type, the inventors combined the developed exposure algorithm with publicly available algorithms to deconvolve biomarkers to tissue type for identification of affected organ system from non-invasive samples.

An example analytics pipeline developed for mRNA is described herein. The same process was applied to miRNA and methylation with minor variations in data preprocessing.

To build an exposure anomaly detection model, publicly available gene expression data was collected from the Genotype Tissue Expression (GTEx) Project, The Cancer Genome Atlas (TCGA) Pan-Cancer analysis project, and the Encyclopedia of DNA Elements (ENCODE) data portal. To ensure that mRNA data collected from repositories recapitulated literature findings of mRNA tissue specific biomarker expression, the data was aggregated across organ systems and TCGA and GTEx repositories for healthy individuals. Using unsupervised clustering of gene expression values for genes with the most variable expression across samples, organ systems could be distinguished by distinct patterns of gene expression.

From both repositories there were clusters of genes with high expression in each tissue that are specific to that organ. Gene biomarkers were also curated from literature that were described as tissue specific, and expression of curated genes was compared across samples. Congruent with literature results, gene expression of biomarkers was higher in organs described to uniquely express those particular biomarkers. In summary, analysis of biomarker gene expression datasets collected from public repositories identified tissue specificity in biomarker expression patterns that can be subsequently used to identify tissue of origin from biological samples.

Data were then normalized to remove batch effects through a combination of quantile and surrogate variable analysis methods. To compare across organ types and repositories, data normalization and batch correction were used to remove technical variation due to experimental conditions, instruments, and personnel.

To identify strategies that allow for data aggregation, several methods of normalization and batch removal were tested with gene expression data. Initial methods tested normalization on TCGA data aggregated across organ systems. For this dataset, quantile normalization was applied to the dataset based on organ system. Quantile normalization ensures that each dataset has a similar distribution. Principal component analysis (PCA) of unnormalized gene expression counts showed that organ systems could not clearly be distinguished using raw data (see FIG. 3). After applying quantile normalization to the data, PCA analysis found consistent variance in the data supporting that normalization does not affect underlying data structure. Following normalization, samples clustered by organ site. The kidney samples still formed a distinct cluster, however, other sample types aggregated by sample type including lung, uterus, bladder, cervix, and prostate samples, highlighting that this method allows for detection of biomarkers that differ in expression pattern between each organ site (see FIG. 4). Accordingly, normalization of mRNA data according to embodiments described herein allows for comparison of samples across disparate datasets.

During experimentation, quantile normalization worked well for samples collected within a repository; however, other technical sources of variation could not be removed with this method alone when comparing samples across repositories. To address limitations of quantile normalization, an additional method of normalization, surrogate variable analysis (SVA) was applied to the data. SVA accounts for hidden sources of variation by introducing new or surrogate variables to remove confounding variation caused by biological sources of variation that are not of interest such as gene level polymorphisms, age of person, sex, and other hidden variables. For this particular experimental analysis, healthy lung tissue and lung related exposures were collected from the Gene Expression Omnibus, Encode, GTEx, and TCGA repository. As a result of PCA of the unnormalized counts, the raw data separated by repository and not by biological variation. But after normalization and applying SVA, batch effects were successfully removed from the data. Sources of biological variability were attributed to the status of the lung tissue. For example, smoking and asthma data, while separate, clustered more closely to each other as compared to healthy.

### Algorithm for Anomaly Detection

In the experimental analysis, an autoencoder algorithm was applied to curated gene expression data from lung samples to classify data into categories of normal or exposed. Samples were classified with 76% accuracy. As discussed above, the dataset was composed of data from four repositories: Encode, GSE201955, GTEx, and TCGA, and the samples corresponded to healthy, cancer, and asthma. These datasets had 17875 features corresponding to different levels of gene activation. In order to train an anomaly detection tool, the data from Encode and GTEx was used for training and GSE201955 and TCGA was reserved for testing.

As there were known noise effects from various external sources, data were normalized using mean and standard deviation from normal samples across repositories. The mean and standard deviation of each of the 17875 features was computed for the healthy data. Features that had a standard deviation of 0 in the training data were discarded, resulting in 16432 features remaining. Features with a standard deviation of zero in the test dataset were set to 1e-5. Then, all data were z-score normalized using the mean and standard deviation of the healthy data for the respective repository.

The model used for this task was an autoencoder with a multitask classifier for repository classification. To help control for batch effects, the autoencoder was trained to create a compressed version of the data and then reconstruct it with as little error as possible, in addition to correctly identifying the repository it was from. The encoder was composed of 4 layers. Each progressing layer contained a linear layer followed by a rectified linear unit (ReLU) and included 1024 nodes, 52 nodes, 128 nodes, and 64 nodes respectively. The decoder was composed of 3 linear layers+ReLU and included 128 nodes, 512 nodes, 1024 nodes. The final decoder layer contained a linear layer with 16432 nodes. The output from the decoder layer containing 512 nodes with ReLU activation was used as weight inputs for a linear layer with a softmax activation. In this example, R^{num rpos} included four nodes, one for each repository. The number of nodes in this layer would increase with increasing number of repositories.

The model was trained for 20 epochs with a batch size of 64 using the Adam optimizer with a loss of 0.001. The mean squared reconstruction error was then calculated for the training data and transformed using a log 10 transformation and using a normal distribution a threshold for healthy-vs-anomalous was chosen using a threshold of a of 0.0025. Therefore, any reconstruction error that exceeded the upper bound of a 99.5% confidence interval was considered to be anomalous.

Training on healthy data resulted in a threshold for sample measurements to determine exposed vs. normal (see FIG. 7). Performance resulted in accuracy of cancer test data at 100.0%, asthma test data at 42.3%, healthy test data at 70%. Overall accuracy on average was 76%. A breakdown of predicted healthy and anomalous can be seen in the following table:

| | Predicted | Predicted |
|---|---|---|
| | Healthy | Anomalous |
| Truth - Healthy GSE201955 | 28 | 18 |
| Truth - Healthy TCGA | 42 | 12 |
| Truth - Asthma | 15 | 11 |
| Truth - Cancer | 0 | 50 |

During the conducted experiment, organ specific patterns of gene expression were successfully characterized, statistical techniques to combine datasets were determined while preserving biological variability, and signatures of healthy and exposure data were identified. Further, an anomaly detection model to identify exposed samples from normal samples was trained and tested on public data with success.

### Example Computer System

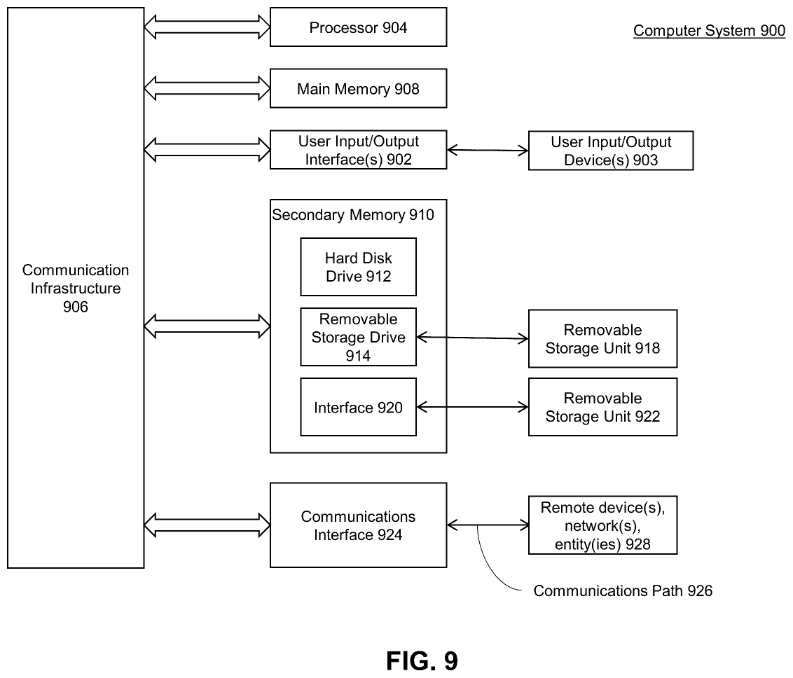

FIG. 9 depicts an example computer system useful for implementing various embodiments. Various embodiments may be implemented across, executed by, and/or deployed in a cloud computing environment containing physical and/or virtual resources and applications (including one or more physical machines, physical storage, virtual machines, virtual storage, or hypervisors). The cloud computing environment may provide computation, software, data access, storage, and/or other services that do not require end-user knowledge of a physical location and configuration of a system and/or a device that delivers the services. For example, one or more computers, networks, or machine learning models used by various embodiments may be hosted in a cloud network. The cloud computing system may include one or more computer resources, such as personal computers, workstations, computers, server devices, or other types of computation and/or communication devices. The cloud resources may include compute instances executing in the cloud computing resources, which may in turn communicate with other cloud computing resources via wired connections, wireless connections, or a combination of wired and wireless connections. Individual users may interact with the cloud network via one or more computer systems in communication with compute resources within the cloud network.

Some embodiments of the generalized metadata generation training pipeline described herein may be hosted by one or more servers coupled to the cloud for distributed execution and/or storage of the pipeline. Some embodiments may be implemented, for example, using one or more well-known computer systems, such as computer system 900 shown in FIG. 9. One or more computer systems 900 may be used, for example, to implement any of the embodiments discussed herein, as well as combinations and sub-combinations thereof.

Computer system 900 may include one or more processors (also called central processing units, or CPUs), such as a processor 904. Processor 904 may be connected to a communication infrastructure or bus 906.

Computer system 900 may also include input/output device(s) 902, such as monitors, keyboards, pointing devices, etc., which may communicate with communication infrastructure 906 through customer input/output interface(s) 902.

One or more of processors 904 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 900 may also include a main or primary memory 908, such as random access memory (RAM). Main memory 908 may include one or more levels of cache. Main memory 908 may have stored therein control logic (i.e., computer software) and/or data.

Computer system 900 may also include one or more secondary storage devices or memory 910. Secondary memory 910 may include, for example, a hard disk drive 912 and/or a removable storage device or drive 914. Removable storage drive 914 may be a floppy disk drive, a magnetic tape drive, a compact disk drive, an optical storage device, tape backup device, and/or any other storage device/drive.

Removable storage drive 914 may interact with a removable storage unit 916. Removable storage unit 916 may include a computer usable or readable storage device having stored thereon computer software (control logic) and/or data. Removable storage unit 916 may be a floppy disk, magnetic tape, compact disk, DVD, optical storage disk, and/any other computer data storage device. Removable storage drive 914 may read from and/or write to removable storage unit 916.

Secondary memory 910 may include other means, devices, components, instrumentalities or other approaches for allowing computer programs and/or other instructions and/or data to be accessed by computer system 900. Such means, devices, components, instrumentalities or other approaches may include, for example, a removable storage unit 922 and an interface 920. Examples of the removable storage unit 922 and the interface 920 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 900 may further include a communication or network interface 924. Communication interface 924 may enable computer system 900 to communicate and interact with any combination of external devices, external networks, external entities, etc. (individually and collectively referenced by reference number 928). For example, communication interface 924 may allow computer system 900 to communicate with external or remote devices 928 over communications path 926, which may be wired and/or wireless (or a combination thereof), and which may include any combination of LANs, WANs, the Internet, etc. Control logic and/or data may be transmitted to and from computer system 900 via communication path 926.

Computer system 900 may also be any of a personal digital assistant (PDA), desktop workstation, laptop or notebook computer, netbook, tablet, smart phone, smart watch or other wearable, appliance, part of the Internet-of-Things, and/or embedded system, to name a few non-limiting examples, or any combination thereof.

Computer system 900 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 900 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML Customer Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 900, main memory 908, secondary memory 910, and removable storage units 916 and 922, as well as tangible articles of manufacture embodying any combination of the foregoing. Such control logic, when executed by one or more data processing devices (such as computer system 900), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 9. In particular, embodiments can operate with software, hardware, and/or operating system implementations other than those described herein.

It is to be appreciated that the Detailed Description section, and not any other section, is intended to be used to interpret the claims. Other sections can set forth one or more but not all exemplary embodiments of the present disclosure as contemplated by the inventor(s), and thus, are not intended to limit the present disclosure and the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

What is claimed is:

1. A computer implemented method for processing an unlabeled omics feature vector, comprising:
receiving, by one or more processors, a plurality of datasets containing omics feature vectors;
preprocessing, by the one or more processors, the plurality of datasets to produce a preprocessed dataset, wherein the preprocessing comprises:
removing technical noise across the plurality of datasets using one or more normalization techniques to produce a corresponding plurality of denoised datasets; and
aggregating the plurality of denoised datasets to generate the preprocessed dataset;
training, by the one or more processors, a machine learning model using a subset of preprocessed omics feature vectors labeled as non-anomalous to perform anomaly detection;
providing, by the one or more processors, an unlabeled omics feature vector to the trained machine learning model;
generating, using the trained machine learning model, a low-dimensional latent space representation of the omics feature vector;
reconstructing, using the trained machine learning model, the omics feature vector from the low-dimensional latent space representation to produce a reconstructed feature vector;
evaluating a reconstruction error between the omics feature vector and the reconstructed feature vector against an anomaly threshold to obtain a reconstruction error evaluation;
generating a feature label indicating whether the omics feature vector is anomalous or non-anomalous based on the reconstruction error evaluation; and
assigning the feature label to the omics feature vector as metadata.

2. The computer implemented method of claim 1, wherein the preprocessing further comprises:
labeling the omics feature vectors in each of the plurality of datasets as anomalous or non-anomalous; and
separating the preprocessed dataset into the subset of preprocessed omics feature vectors labeled as non-anomalous and a subset of preprocessed omics feature vectors labeled as anomalous.

3. The computer implemented method of claim 1, wherein the preprocessing further comprises:
confirming that the technical noise across the plurality of datasets has been removed using principal component analysis.

4. The computer implemented method of claim 1, wherein the subset of preprocessed omics feature vectors labeled as non-anomalous comprises the set of omics feature vectors across each of the plurality of denoised datasets that are identified to not be associated with a disease, an illness, or adverse health symptoms.

5. The computer implemented method of claim 1, wherein the one or more normalization techniques comprise quantile normalization, surrogate variable estimation, or z-score normalization, or combinations thereof.

6. The computer implemented method of claim 1, wherein the omics feature vectors comprise gene expression levels or methylation statuses of mRNA, miRNA, methylated DNA, or microbiomes.

7. The computer implemented method of claim 1, wherein the machine learning model is an autoencoder or a convolutional neural network.

8. The computer implemented method of claim 1, wherein the plurality of datasets are public repository datasets or generated datasets.

9. A system, comprising:
one or more memories;
at least one processor each coupled to at least one of the memories and configured to perform operations comprising:
receiving a plurality of datasets containing omics feature vectors;
preprocessing the plurality of datasets to produce a preprocessed dataset, wherein the preprocessing comprises:
removing technical noise across the plurality of datasets using one or more normalization techniques to produce a corresponding plurality of denoised datasets; and
aggregating the plurality of denoised datasets to generate the preprocessed dataset;
training a machine learning model using a subset of preprocessed omics feature vectors labeled as non-anomalous to perform anomaly detection;
providing an unlabeled omics feature vector to the trained machine learning model;
generating, using the trained machine learning model, a low-dimensional latent space representation of the omics feature vector;
reconstructing, using the trained machine learning model, the omics feature vector from the low-dimensional latent space representation to produce a reconstructed feature vector;
evaluating a reconstruction error between the omics feature vector and the reconstructed feature vector against an anomaly threshold to obtain a reconstruction error evaluation;
generating a feature label indicating whether the omics feature vector is anomalous or non-anomalous based on the reconstruction error evaluation; and
assigning the feature label to the omics feature vector as metadata.

10. The system of claim 9, wherein the preprocessing further comprises:
labeling the omics feature vectors in each of the plurality of datasets as anomalous or non-anomalous; and
separating the preprocessed dataset into the subset of preprocessed omics feature vectors labeled as non-anomalous and a subset of preprocessed omics feature vectors labeled as anomalous.

11. The system of claim 9, wherein the preprocessing further comprises:
confirming that the technical noise across the plurality of datasets has been removed using principal component analysis.

12. The system of claim 9, wherein the subset of preprocessed omics feature vectors labeled as non-anomalous comprises the set of omics feature vectors across each of the plurality of denoised datasets that are identified to not be associated with a disease, an illness, or adverse health symptoms.

13. The system of claim 9, wherein the one or more normalization techniques comprise quantile normalization, surrogate variable estimation, or z-score normalization, or combinations thereof.

14. The system of claim 9, wherein the omics feature vectors comprise gene expression levels or methylation statuses of mRNA, miRNA, methylated DNA, or microbiomes.

15. The system of claim 9, wherein the machine learning model is an autoencoder or a convolutional neural network.

16. The system of claim 9, wherein the plurality of datasets are public repository datasets or generated datasets.

17. A non-transitory computer-readable medium having instructions stored thereon that, when executed by at least one computing device, causes the at least one computing device to perform operations comprising:
receiving a plurality of datasets containing omics feature vectors;
preprocessing the plurality of datasets to produce a preprocessed dataset, wherein the preprocessing comprises:
removing technical noise across the plurality of datasets using one or more normalization techniques to produce a corresponding plurality of denoised datasets; and
aggregating the plurality of denoised datasets to generate the preprocessed dataset;
training a machine learning model using a subset of preprocessed omics feature vectors labeled as non-anomalous to perform anomaly detection;
providing an unlabeled omics feature vector to the trained machine learning model;
generating, using the trained machine learning model, a low-dimensional latent space representation of the omics feature vector;
reconstructing, using the trained machine learning model, the omics feature vector from the low-dimensional latent space representation to produce a reconstructed feature vector;
evaluating a reconstruction error between the omics feature vector and the reconstructed feature vector against an anomaly threshold to obtain a reconstruction error evaluation;
generating a feature label indicating whether the omics feature vector is anomalous or non-anomalous based on the reconstruction error evaluation; and
assigning the feature label to the omics feature vector as metadata.

18. The non-transitory computer-readable medium of claim 17, wherein the preprocessing further comprises:
labeling the omics feature vectors in each of the plurality of datasets as anomalous or non-anomalous; and
separating the preprocessed dataset into the subset of preprocessed omics feature vectors labeled as non-anomalous and a subset of preprocessed omics feature vectors labeled as anomalous.

19. The non-transitory computer-readable medium of claim 17, wherein the preprocessing further comprises:
confirming that the technical noise across the plurality of datasets has been removed using principal component analysis.

20. The non-transitory computer-readable medium of claim 17, wherein the subset of preprocessed omics feature vectors labeled as non-anomalous comprises the set of omics feature vectors across each of the plurality of denoised datasets that are identified to not be associated with a disease, an illness, or adverse health symptoms.
